(19) United States
(12) Patent Application Publication
(10) Pub. No.: US 2024/0404683 A1
(43) Pub. Date: Dec. 5, 2024

(54) MEDICAL INFORMATION PROCESSING APPARATUS, MEDICAL INFORMATION PROCESSING SYSTEM, AND MEDICAL INFORMATION PROCESSING METHOD

(71) Applicant: CANON MEDICAL SYSTEMS CORPORATION, Otawara-shi (JP)

(72) Inventors: Yuto TAKAHASHI, Otawara (JP); Shiro KATO, Utsunomiya (JP); Kyohei NORO, Nasushiobara (JP)

(73) Assignee: CANON MEDICAL SYSTEMS CORPORATION, Otawara-shi (JP)

(21) Appl. No.: 18/675,470

(22) Filed: May 28, 2024

(30) Foreign Application Priority Data
May 30, 2023 (JP) 2023-088628

(51) Int. Cl. G16H 40/20; G16H 30/20; G16H 50/70

(52) U.S. Cl. CPC G16H 40/20; G16H 30/20; G16H 50/70

(57) ABSTRACT

In one embodiment, a medical information processing apparatus includes first processing circuitry. The first processing circuitry acquires attribute information indicating an attribute of an object and analysis information indicating an analysis result of a medical image of the object, generates a pre-order including at least one of an imaging order and a treatment order based on the attribute information and the analysis information, and causes a display to display the pre-order.

### CROSS-REFERENCE TO RELATED APPLICATION

This application is based upon and claims the benefit of priority from Japanese Patent Application No. 2023-088628, filed on May 30, 2023, the entire contents of which are incorporated herein by reference.

### FIELD

Disclosed embodiments relate to a medical information processing apparatus, a medical information processing system, and a medical information processing method.

### BACKGROUND

An automatic analysis apparatus for medical images acquired by imaging an object supports a flow of “imaging (examination), subsequent analysis, subsequent result provision” as a workflow for each examination of CT image analysis and/or MR (Magnetic Resonance) image analysis regarding, for example, cerebral infarction. In addition, the automatic analysis apparatus has a function to notify another system of completion of analysis and findings (e.g., presence/absence of abnormality) regarding the analysis result of the medical images.

However, in the automatic analysis apparatus, though the above-described flow is supported as a workflow for each examination, a workflow from imaging to treatment and a workflow spanning a plurality of examinations are not supported. Thus, the attending doctor needs to create an order from scratch for giving instruction about the next treatment on the basis of the analysis result from the automatic analysis apparatus. In some case, the time required for generating the order becomes the bottleneck, which makes it impossible to provide faster examination and treatment for the object.

### BRIEF DESCRIPTION OF THE DRAWINGS

In the accompanying drawings:

FIG. 1 is a block diagram illustrating a configuration of a medical information processing system according to the first embodiment;

FIG. 2 is a schematic diagram illustrating a data flow in the medical information processing system according to the first embodiment;

FIG. 3 is a flowchart illustrating processing to be executed by the medical information processing system according to the first embodiment;

FIG. 4A is a schematic diagram illustrating a configuration of an object information table;

FIG. 4B is a schematic diagram illustrating a configuration of a pre-order generation table;

FIG. 5A is a schematic diagram illustrating a configuration of a contraindication table;

FIG. 5B is a schematic diagram illustrating a configuration of the pre-order generation table;

FIG. 6 is a schematic diagram illustrating a screen that presents a plurality of workflows according to the first embodiment;

FIG. 7 is a schematic diagram illustrating a screen that presents the reason for the workflow according to the first embodiment;

FIG. 8 is a schematic diagram illustrating a pre-order generation screen according to the first embodiment;

FIG. 9 is a schematic diagram illustrating another pre-order generation screen according to the first embodiment; and

FIG. 10 is a schematic diagram illustrating a proposed workflow regarding an object with chest pain according to the second embodiment.

### DETAILED DESCRIPTION

Hereinbelow, a detailed description will be given of embodiments of a medical information processing apparatus, a medical information processing system, and a medical information processing method by referring to the accompanying drawings.

In one embodiment, a medical information processing apparatus includes first processing circuitry. The first processing circuitry acquires attribute information indicating an attribute of an object and analysis information indicating an analysis result of a medical image of the object, generates a pre-order including at least one of an imaging order and a treatment order based on the attribute information and the analysis information, and causes a display to display the pre-order.

### Prior Art and Embodiment

The medical information processing apparatus 2 receives an input of an image depicting an object, inputs the image to at least one necessary analysis application selected from among many image analysis applications in an analysis server (each image is often analyzed by a plurality of applications), outputs the analysis result to the server of a medical picture archiving and communication system (PACS) in such a manner that a doctor in charge of image interpretation (hereinafter referred to as an image interpretation doctor) can view the image.

Conventionally, when an image of an object is inputted, the doctor checks the diagnostic purpose of the object and the type of image, and then manually selects an image-analysis application one by one on the basis of the checked result so as to analyze the inputted image by using the selected application.

Contrastively, in the present embodiment, the doctor preliminarily or previously designates execution rules regarding the types of the input image and diagnosis as well as the analysis application (or a group of applications) to be used for analysis in the medical information processing apparatus 2. The doctor defines the analysis process of the image analysis application on the basis of the designated rules, and causes the medical information processing apparatus 2 to automatically execute the analysis process.

In this configuration, the doctor can receive the analysis result of the necessary analysis application by inputting an image into the medical information processing apparatus 2 without having to make any setting such as designation of an analysis application.

### First Embodiment

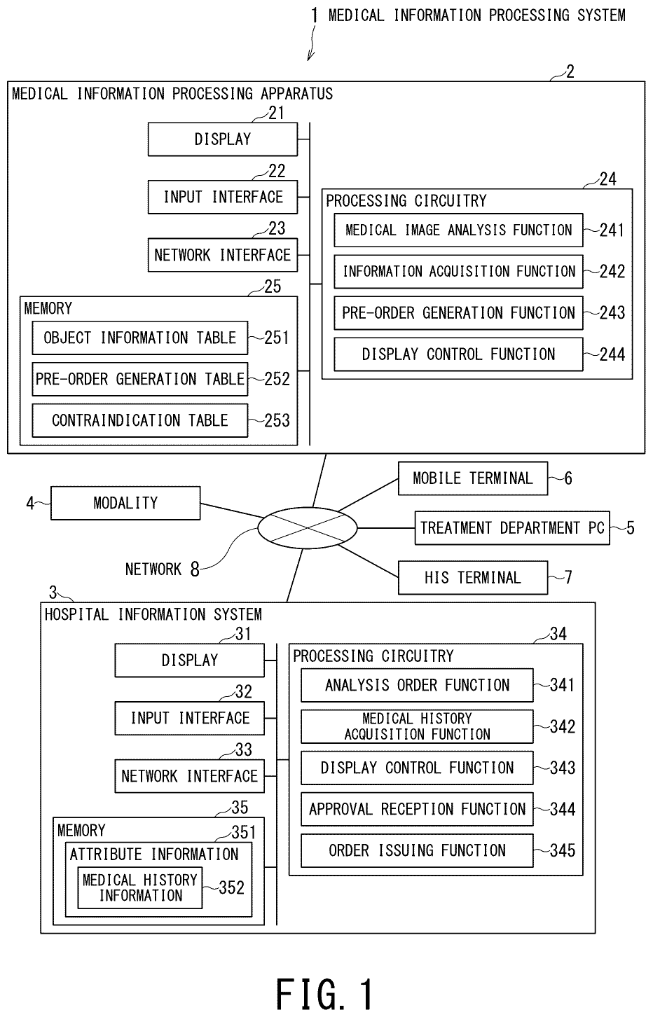

FIG. 1 is a block diagram illustrating a configuration of a medical information processing system 1 according to the first embodiment. As shown in FIG. 1, the medical information processing system 1 includes the medical information processing apparatus 2, a hospital information system 3, a modality 4, a treatment department PC 5, a mobile terminal 6 (i.e., portable terminal 6), and a HIS (hospital information system) terminal 7. The respective components are configured to be able to communicate with each other via a network 8. In the following, it is assumed that data are transmitted and received between respective components via the network 8, and the phrase “via the network 8” is omitted in description of transmission/reception of data.

Hereinafter, terms to be used in the embodiments will be described. A workflow is a process of medical treatment for an object such as a patient. For example, the workflow is a series of steps of “examination, subsequent analysis, and subsequent treatment”. An order is an instruction given by a doctor that indicates medical treatment to be performed on an object. A pre-order is a choice or an option for an order. In some cases, the pre-order are issued as an order without any change. In some cases, the contents of the pre-order are modified (including additions and deletions) and then it is issued as an order.

Aspects of an order include an analysis order, an imaging order, and a treatment order. The analysis order is an order related to analysis of a medical image acquired by imaging an object, and includes designation of an analysis application to be used for analysis of a medical image obtained from a modality, for example. The imaging order is an order related to imaging of an object and includes, for example, an instruction for imaging procedure, such as injection of a contrast medium. The treatment order is an order related to treatment of an object and includes drug usage for treatment and symptom control.

Next, each component of the medical information processing system 1 will be described. The medical information processing apparatus 2 receives attribute information indicating attributes of the object from the hospital information system 3, and receives analysis information indicating an analysis result of the medical image generated by imaging the object from the modality 4. The medical information processing apparatus 2 generates a pre-order on the basis of the received attribute information of the object and the received analysis information of the object, and transmits the pre-order to the hospital information system 3. In other words, the medical information processing apparatus 2 generates the pre-order in cooperation with the hospital information system 3.

In detail, the medical information processing apparatus 2 uses the attribute information of the object and the analysis information of the object to predict a future workflow. The medical information processing apparatus 2 then generates a pre-order that is a choice of a necessary order based on the workflow, and transmits the pre-order to the hospital information system 3. Note that the medical information processing apparatus 2 may acquire the medical image of the object and analyze the medical image.

The hospital information system 3 is abbreviated as HIS and enables operation, storage, and viewing of electronic medical records and in-hospital data of respective systems, for example. The respective systems include a medical accounting system (i.e., receipt computer), a pharmacy management system, a medical image management system, a reservation system, an admission/discharge management system (i.e., hospitalization/release management system), and a hospital-meal/nutrition management system, for example. The hospital information system 3 is composed of a plurality of general information processing apparatuses such as a personal computer and a workstation. The hospital information system 3 is one example of a hospital information management system.

In detail, the hospital information system 3 stores information necessary for determining pre-order generation and order issuance, and transmits this information to the medical information processing apparatus 2. Further, the hospital information system 3 receives the pre-order from the medical information processing apparatus 2 and causes the HIS terminal 7 to display the pre-order.

The doctor issues the order by checking and approving the pre-order generated by the medical information processing apparatus 2 on the HIS terminal 7. In this case, the hospital information system 3 acquires the pre-order approved by the doctor from the HIS terminal 7, and applies the pre-order to transmit the imaging order to the modality 4 or transmit the treatment order to the treatment department PC 5.

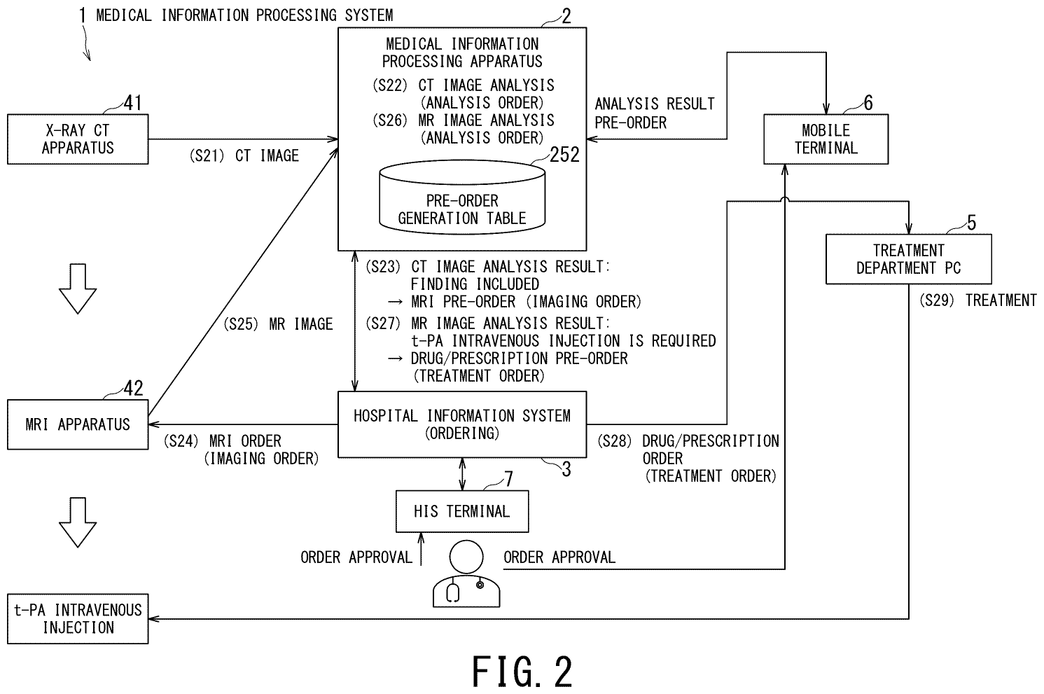

The modality 4 is an examination apparatus, such as an X-ray CT (Computed Tomography) apparatus 41 and an MRI (Magnetic Resonance Imaging) apparatus 42 (FIG. 2) installed in an examination room, which generates an examination image of an object, and transmits the examination image to the medical information processing apparatus 2. In response to receipt of the imaging order from the hospital information system 3 or receiving another trigger, the modality 4 provides a display for instructing the user to perform operation on imaging of the object.

The treatment department PC 5 is installed in a treatment room, and receives the treatment order from the hospital information system 3 so as to provides a display for giving instructions about prescribing drugs and treating the object.

The mobile terminal 6 is a terminal for a doctor to check and approve the pre-order generated by the medical information processing apparatus 2. The mobile terminal 6 is a terminal such as a smartphone that communicates with the medical information processing apparatus 2.

The HIS terminal 7 is a terminal for a doctor to check and approve the pre-order generated by the medical information processing apparatus 2. The HIS terminal 7 is a dedicated terminal that communicates with the hospital information system 3.

As shown in FIG. 1, the medical information processing apparatus 2 includes a display 21, an input interface 22, a network interface 23, processing circuitry 24, and a memory 25.

The display 21 is composed of a general display output device such as a liquid crystal display and an OLED (Organic Light Emitting Diode) display, for example. The input interface 22 is composed of general input devices such as a trackball, a switch button, a mouse, a keyboard, and a numeric keypad, and a touch panel, and outputs an operation input signal corresponding to the doctor's operation to the processing circuitry 24.

The network interface 23 implements various information communication protocols according to the type of the network 8. The network interface 23 communicably connects the medical information processing apparatus 2 to the hospital information system 3, the modality 4, and the mobile terminal 6 via the network 8 on the basis of these various protocols.

The processing circuitry 24 implements a function of integrally controlling the medical information processing apparatus 2. In addition, the processing circuitry 24 is a processor that reads out and executes programs stored in the memory 25 so as to analyze medical images and issue pre-orders. The processing circuitry 24 is one example of first processing circuitry. The processor of the processing circuitry 24 implements a medical image analysis function 241, an information acquisition function 242, a pre-order generation function 243, and a display control function 244 by reading out and executing the computer programs stored in the memory 25 or a memory in the processing circuitry 24.

The medical image analysis function 241 includes a function to analyze the medical image of the object depending on the analysis order received from the hospital information system 3 and output the analysis result.

The information acquisition function 242 includes a function to: acquire attribute information indicating attributes of the object from the hospital information system 3; and acquire analysis information indicating the analysis result of the medical image generated by imaging the object from the medical image analysis function 241.

The pre-order generation function 243 includes a function to generate the pre-order including at least one of the imaging order and the treatment order on the basis of both the analysis information of the object and the attribute information of the object acquired by the information acquisition function 242.

The display control function 244 includes a function to display the pre-order generated by the pre-order generation function 243 on the mobile terminal 6.

The mobile terminal 6 is one example of a display. The analysis information is an analysis result obtained by analyzing a medical image with the use of an image analysis application or a report made by an image interpretation doctor on the basis of the analysis result.

The memory 25 includes a recording medium that can be read by a processor, as exemplified by: a semiconductor memory element such as a flash memory and a RAM (Random Access Memory); a hard disk; and an optical disk. The memory 25 stores programs and parameter data to be used by the processing circuitry 24, for example.

The memory 25 stores at least an object information table 251, a pre-order generation table 252, and a contraindication table 253. The object information table 251 is composed of information in the hospital information system 3 and the past execution history of the pre-order generation table 252. The pre-order generation table 252 is generated from the object information table 251, and is a table in which workflows are arranged in order of likelihood of execution. The contraindication table 253 is generated from information in the hospital information system 3, and includes information on contraindications, allergies, and other special remarks of each object. Details of the object information table 251, the pre-order generation table 252, and the contraindication table 253 will be described separately by using configuration cases.

As shown in FIG. 1, the hospital information system 3 includes a display 31, an input interface 32, a network interface 33, processing circuitry 34, and a memory 35.

The display 31 is composed of a general display output device such as a liquid crystal display and an OLED display, for example. The input interface 32 is composed of general input devices such as a trackball, a switch button, a mouse, a keyboard, and a numeric keypad. The input interface 32 outputs an operation input signal corresponding to the doctor's operation to the processing circuitry 34. The network interface 33 implements various information communication protocols according to the type of the network 8. The network interface 33 communicably connects the hospital information system 3 to the medical information processing apparatus 2, the modality 4, the treatment department PC 5, and the HIS terminal 7 via the network 8 on the basis of these various protocols.

The processing circuitry 34 implements a function of integrally controlling the hospital information system 3. The processing circuitry 34 is a processor that reads out and executes programs stored in the memory 35 so as to perform processing for allowing the in-hospital data to be operated, stored, and viewed. The processing circuitry 34 is one example of second processing circuitry. The processor of the processing circuitry 34 implements an analysis order function 341, a medical history acquisition function 342, a display control function 343, an approval reception function 344, and an order issuing function 345 by reading out and executing the computer programs stored in the memory 35 or a memory in the processing circuitry 34.

The analysis order function 341 includes a function to transmit the analysis order, which is an instruction to analyze the medical image of the object and output the analysis result, to the medical information processing apparatus 2.

The medical history acquisition function 342 includes a function to acquire the medical history information indicating a medical record (i.e., anamnesis or clinical record) of the object from the memory 35.

The display control function 343 includes a function to: acquire the pre-orders from the medical information processing apparatus 2; narrow down the pre-orders on the basis of the medical history information in the memory 35; and display the narrowed-down pre-order on the HIS terminal 7. The HIS terminal 7 is one example of the display.

The approval reception function 344 includes a function to receive approval of the pre-order by a doctor's operation on the HIS terminal 7. A doctor is one example of a user.

The order issuing function 345 includes a function to issue at least one of the imaging order and the treatment order on the basis of the pre-order of which the approval has been received by the approval reception function 344.

The memory 35 includes a recording medium that can be read by a processor, as exemplified by: a semiconductor memory element such as a flash memory and a RAM; a hard disk; and an optical disk. The memory 35 stores programs and parameter data to be used by the processing circuitry 34, for example.

The memory 35 stores at least attribute information 351 and medical history information 352. The attribute information 351 includes ID, age, height, weight, allergies, double medication, contraindications, radiation doses, and the past medical history of the object. The medical history information 352 includes allergies, double medication, contraindications, radiation doses, and the past medical history of the object. The medical history information 352 is used to make a decision regarding order issuance (in particular, extraction of valid pre-orders for the object). Note that the attribute information 351 includes the medical history information 352. In other words, the medical history information 352 is part of the attribute information 351.

FIG. 2 is a schematic diagram illustrating a data flow in the medical information processing system 1 according to the first embodiment. This data flow particularly shows the flow of data from determination of necessity of thrombolysis (t-PA) for cerebral infarction to treatment.

As a premise, the flow of “CT image analysis, subsequent MR image analysis, subsequent t-PA intravenous injection” is interpreted as one workflow. The medical information processing apparatus 2 uses the results of CT image analysis and MR image analysis and the object attribute information from the hospital information system 3 for issuing the pre-order. This configuration reduces the time and effort by the doctor to generate the order.

The following two workflows are presented to the doctor as possible or conceivable workflows based on the attribute information of the object and the analysis information of the object: (1) t-PA intravenous injection; and (2) MRI and subsequent t-PA intravenous injection. When the doctor selects one workflow, the pre-order is generated on the basis of the selected workflow. Afterward, when the doctor approves the pre-order, the formal order is easily issued. Hereinbelow, its details will be described.

In the step S21, in response to receiving a predetermined trigger, the X-ray CT apparatus 41 generates CT images of the object by imaging the object and transmits the data of the CT images to the medical information processing apparatus 2.

In the step S22, the medical information processing apparatus 2 receives the CT image data of the object from the X-ray CT apparatus 41, and analyzes the CT images by using a dedicated analysis application. Note that the medical information processing apparatus 2 may cause the analysis application to perform analysis using a model of AI (Artificial Intelligence) or using a normal program.

In the step S23, as a result of the CT image analysis, the medical information processing apparatus 2 acquires information indicating that “there is a finding of cerebral infarction, but it is difficult to determine necessity of the t-PA intravenous injection, so MR images are required.” Accordingly, the medical information processing apparatus 2 transmits an MRI pre-order to the hospital information system 3 as the imaging order.

In the step S24, the hospital information system 3 receives the MRI pre-order from the medical information processing apparatus 2 and transmits the MRI pre-order to the HIS terminal 7. In response to receipt of approval of the MRI pre-order from the HIS terminal 7, the hospital information system 3 transmits the MRI order to the MRI apparatus 42 as the imaging order.

In the step S25, the MRI apparatus 42 receives the MRI order from the hospital information system 3, and images the object so as to generate MR images. The MRI apparatus 42 transmits the data of the MR images to the medical information processing apparatus 2.

In the step S26, the medical information processing apparatus 2 receives the data of the MR images of the object from the MRI apparatus 42 and analyzes the MR images by using a dedicated analysis application. Note that the medical information processing apparatus 2 may cause the analysis application to perform analysis using a model of AI or using a normal program.

In the step S27, as a result of analyzing the MR images, the medical information processing apparatus 2 acquires information indicating that “the t-PA intravenous injection is necessary”. Accordingly, the medical information processing apparatus 2 generates a drug/prescription pre-order as the treatment order and transmits it to the hospital information system 3. In order to prescribe a drug, information on allergies, double medication, contraindications, and/or other special notes of the object is required.

In the step S28, the hospital information system 3 receives the drug/prescription pre-order from the medical information processing apparatus 2 and transmits the drug/prescription pre-order to the HIS terminal 7. The doctor checks the contents of the drug/prescription pre-order displayed on the HIS terminal 7. If there is no problem with the contents, the doctor clicks an approve button, for example. This action completes the operation for approving the drug/prescription pre-order. Subsequently, in response to receipt of the approval of the drug/prescription pre-order from the HIS terminal 7, the hospital information system 3 transmits the drug/prescription order to the treatment department PC 5 as the treatment order.

In the step S29, the treatment department PC 5 receives the drug/prescription order from the hospital information system 3. Subsequently, the treatment department PC 5 displays the contents of the received drug/prescription order on the display so that the p-TA intravenous injection is performed as a treatment for the object, for example.

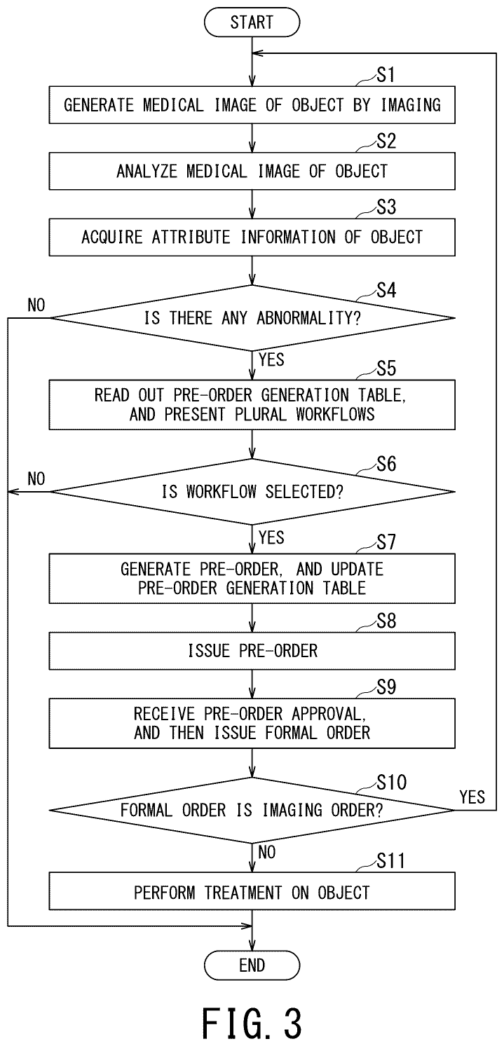

FIG. 3 is a flowchart illustrating processing to be executed by the medical information processing system 1 according to the first embodiment. This flowchart shows the general processing flow of the medical information processing system 1 without specifying the disease name of the object.

In the step S1, in response to receipt of the predetermined trigger or the imaging order from the hospital information system 3, the modality 4 of the medical information processing system 1 images the object to generate medical images of the object. The modality 4 transmits the data of the medical images to the medical information processing apparatus 2.

In the step S2, the network interface 23 of the medical information processing apparatus 2 receives the data of the medical images of the object from the modality 4. The medical image analysis function 241 of the medical information processing apparatus 2 analyzes the data of the medical images.

In the step S3, the information acquisition function 242 of the medical information processing apparatus 2 acquires the attribute information of the object via the network interface 23 from the hospital information system 3.

In the step S4, if there is any abnormality in the finding based on a result of the analysis in the step S2 (YES in the step S4), the processing circuitry 24 of the medical information processing apparatus 2 advances the processing to the step S5. If there is no abnormality in the finding based on a result of the analysis in the step S2 (NO in the step S4), the processing circuitry 24 completes the processing.

In the step S5, the processing circuitry 24 of the medical information processing apparatus 2 reads out the pre-order generation table 252 from the memory 25, and causes the mobile terminal 6 to display a plurality of workflows. The doctor refers to the plurality of workflows displayed on the mobile terminal 6, and selects one of the workflows. The mobile terminal 6 transmits the selection result of the workflows by the doctor to the medical information processing apparatus 2. The selection result includes a case where no workflow is selected.

In the step S6, the network interface 23 of the medical information processing apparatus 2 receives the selection result of the workflows from the mobile terminal 6. If any workflow is selected (YES in the step S6), the processing circuitry 24 advances the processing to the step S7. Conversely, if workflow is not selected (NO in the step S6), the processing circuitry 24 completes the processing.

In the step S7, the pre-order generation function 243 of the medical information processing apparatus 2 generates the pre-order on the basis of the selected workflow, and updates the pre-order generation table 252 of the memory 25 with this generated pre-order. In other words, the pre-order generation function 243 identifies one or plural candidate diseases (i.e., conceivable disease names) of the object on the basis of the attribute information of the object and the analysis information of the object, then acquires the pre-order generation table 252 of (i) the imaging order as an instruction to generate medical images necessary for diagnosing the candidate disease(s) or (ii) the treatment order as an instruction to perform treatment of the candidate disease(s), and then generates the pre-order on the basis of the acquired pre-order generation table 252. The pre-order generation table 252 is one example of history data.

In the step S8, the processing circuitry 24 of the medical information processing apparatus 2 issues the pre-order. The display control function 244 of the medical information processing apparatus 2 may cause the mobile terminal 6 to display the pre-order.

The entire system may also be configured such that: the network interface 23 of the medical information processing apparatus 2 transmits the pre-order to the hospital information system 3; the network interface 33 of the hospital information system 3 receives the pre-order from the medical information processing apparatus 2; and the display control function 343 causes the HIS terminal 7 to display the received pre-order. The entire system may also be configured such that the mobile terminal 6 or the HIS terminal 7 can modify the pre-order before or after issuing the pre-order. According to the steps S7 and S8, each time the pre-order is generated and issued, the issued pre-order is stored in the pre-order generation table 252 as the history data.

In the step S9, the approval reception function 344 of the hospital information system 3 receives the doctor's approval of the pre-order from the mobile terminal 6 or the HIS terminal 7. The approval reception function 344 may receive the approval of the pre-order via the medical information processing apparatus 2 from the mobile terminal 6. Further, the order issuing function 345 of the hospital information system 3 issues the formal order on the basis of the pre-order of which the approval has been received by the reception approval function 344.

In the step S10, if the formal order is the imaging order (YES in the step S10), the order issuing function 345 transmits the imaging order to the modality 4, and the medical information processing system 1 returns the processing to the step S1. If the formal order is not the imaging order (NO in the step S10), the order issuing function 345 transmits the treatment order to the treatment department PC 5, and the medical information processing system 1 advances the processing to the step S11.

In the step S11, the treatment department PC 5 displays the contents of the treatment order on the display so that treatment for the object is performed on the basis of the treatment order received from the hospital information system 3, for example. Afterward, the medical information processing system 1 completes a series of processing.

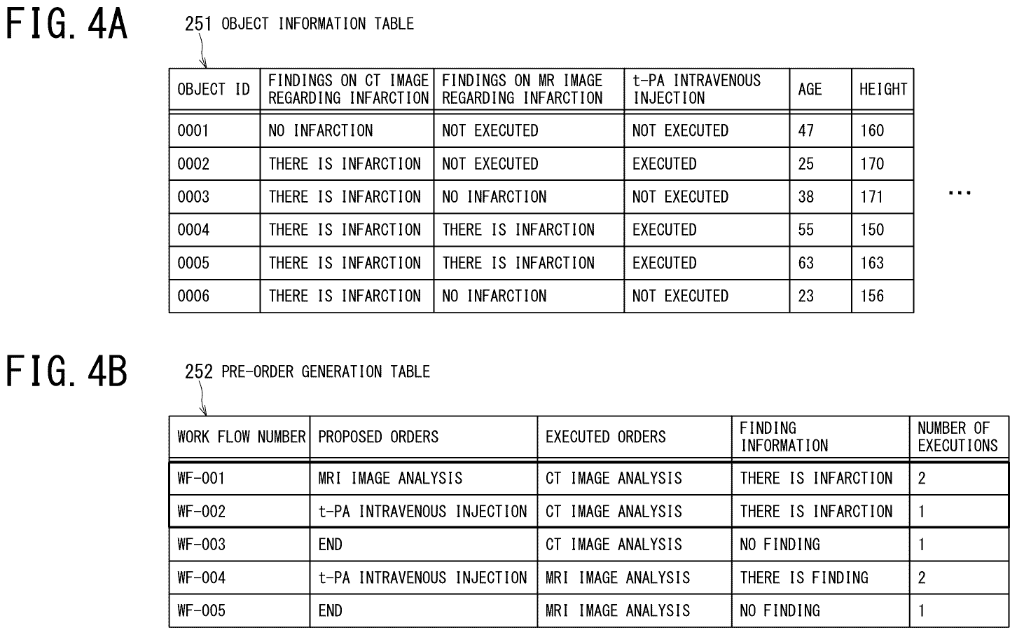

FIG. 4A and FIG. 4B are schematic diagrams illustrating the processing of presenting the workflow according to the first embodiment. FIG. 4A illustrates a configuration of the object information table 251. As shown in FIG. 4A, the object information table 251 is composed of a plurality of records including an object ID, findings on CT images regarding infarction, findings on MR images regarding infarction, t-PA intravenous injection, age, and height. The object ID is a number unique to each object and is used to identify the object. The findings on CT images regarding infarction indicates whether there is any finding of infarction in the CT images of the object or not. The findings on MR images regarding infarction indicates whether there is any finding of infarction in the MR images of the object or whether MRI has been performed. The t-PA intravenous injection indicates whether the t-PA intravenous injection is already performed or not. The age indicates the age of the object. The height indicates the height (in centimeters) of the object.

FIG. 4B illustrate a configuration of the pre-order generation table 252. As shown in FIG. 4B, the pre-order generation table 252 is composed of a plurality of records including workflow numbers, proposed orders, executed orders, finding information, and the number of executions. The workflow number is a specified number given to each workflow, and is used to identify the workflow. The proposed order is an order that is proposed to be executed next. The executed order is the most recently executed order. The finding information indicates whether there is any abnormality in the finding based on the execution result (or the image analysis result) of the executed order or not. The number of executions indicates the number of times the workflow has been executed so far.

The pre-order generation function 243 of the medical information processing apparatus 2 generates the pre-order generation table 252 from the object information table 251, and sorts the records in the pre-order generation table 252 in descending order of the number of executions. This configuration allows workflows to be presented in order of likelihood of execution.

For example, if there is finding of infarction in the CT images of the object being focused on, the pre-order generation function 243 extracts, from the pre-order generation table 252, the records satisfying both conditions including (i) that the executed order is CT image analysis and (ii) that finding information of infarction is included, and then sorts the extracted records in order of the number of executions. The results are the two records shown by the thick-line frame in FIG. 4B.

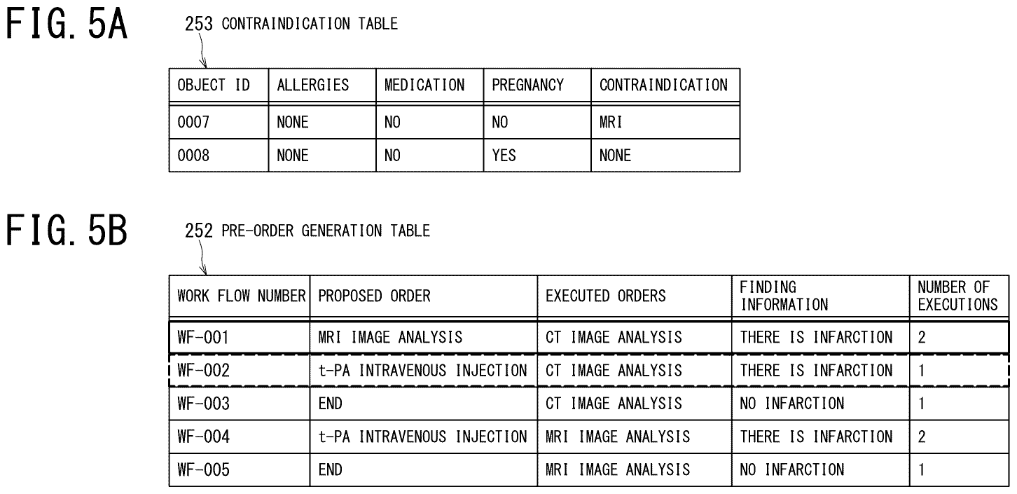

FIG. 5A and FIG. 5B are schematic diagrams illustrating the processing of presenting the workflow according to the first embodiment. FIG. 5A illustrates a configuration of the contraindication table 253. The contraindication table 253 is generated from the attribute information 351 of the hospital information system 3. As shown in FIG. 5A, the contraindication table 253 is composed of a plurality of records including the object ID, allergies, medication, pregnancy, and contraindication. The object ID is a specified number given to each object, and is used to identify the object. The allergies indicate whether the object is allergic to something or not. The medication indicates whether the object is receiving medication or not. The pregnancy indicates whether the object is pregnant or not. The contraindications indicate something that the object detests or something that is prohibited by a doctor, and if there is no such thing, “none” is set in this column.

FIG. 5B illustrate the configuration of the pre-order generation table 252, and its details are described above by using FIG. 4B.

The pre-order generation function 243 of the medical information processing apparatus 2 generates the pre-order generation table 252 from the object information table 251, and sorts the records in the pre-order generation table 252 in descending order of the number of executions. The pre-order generation function 243 then narrows down the records in the pre-order generation table 252 on the basis of the contraindication table 253.

For example, if there is a finding of infarction in the CT images of the object whose object ID is 0007 in FIG. 5A, MRI is contraindicated for the object, and thus the pre-order generation function 243 extracts the record in the thick dashed-line frame, workflow number of which is WF-002, in FIG. 5B. If there is a finding of infarction in the CT images of the object whose object ID is 0008 in FIG. 5A, the object does not have any contraindications, and thus the pre-order generation function 243 extracts the record in the bold solid-line frame, workflow number of which is WF-001, which is the workflow with the highest number of executions in FIG. 5B. This configuration can display executable workflows.

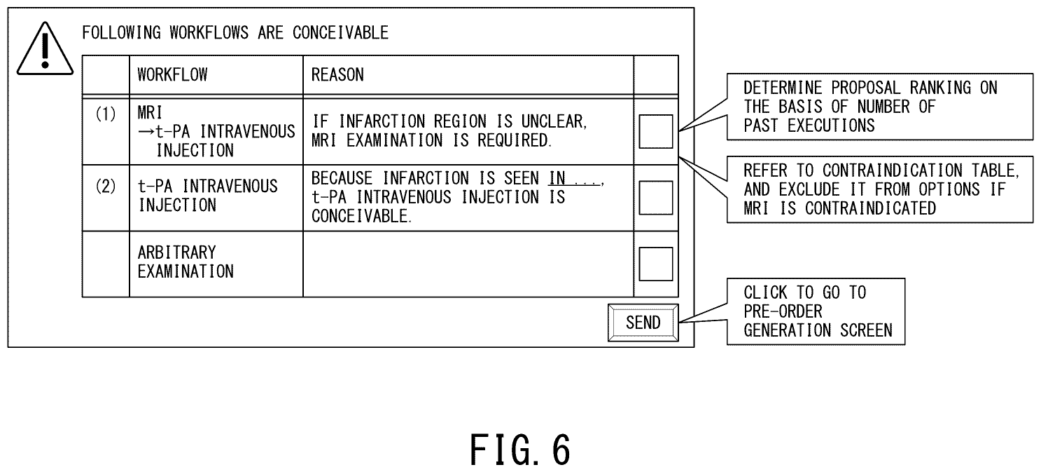

FIG. 6 illustrates a screen that presents a plurality of workflows according to the first embodiment. This screen is displayed on the mobile terminal 6 or the HIS terminal 7 by using a viewer, for example (hereinafter, the same). As shown in FIG. 6, two workflows and the reasons for the respective workflows are displayed. In other words, the pre-order generation function 243 of the medical information processing apparatus 2 further generates the grounds for generating each workflow on the basis of the attribute information of the object and the analysis information of the object. The display control function 244 then displays the generated grounds together with each workflow on the mobile terminal 6 or the HIS terminal 7. The workflow is one example of the pre-order. The reason is one example of rationale information indicating the rationale or grounds for generating the workflow.

Furthermore, an optional or arbitrary examination can be selected in order to handle the case where neither of the two workflows are selected. A checkbox is provided on the rightmost side of the screen, and the user can enter a check mark (tick) to indicate that it has been selected. A send button is provided at the bottom of the screen. When the doctor touches or clicks the send button, the display screen transitions to the pre-order generation screen.

The proposal order of workflows is determined depending on the number of execution times in the past. In other words, the pre-order generation function 243 generates a plurality of workflows. The display control function 244 narrows down the workflows to be displayed on the mobile terminal 6 or the HIS terminal 7 depending on the number of times that each workflow has been executed. The workflow is one example of the pre-order. Referring to the contraindication table 253, for example, if MRI is contraindicated, the workflow of “MRI and the subsequent t-PA intravenous injection” is excluded from the options.

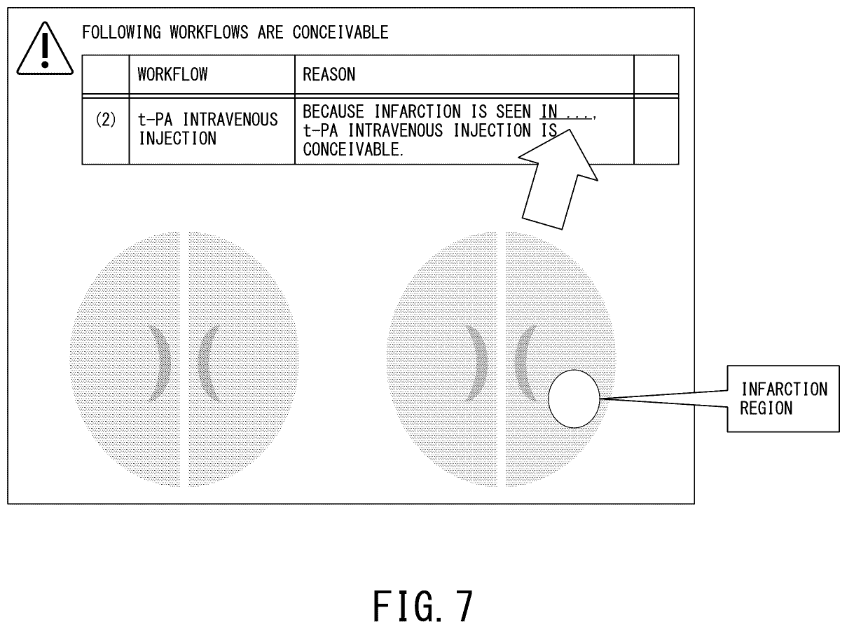

FIG. 7 illustrates a screen that presents the grounds and reason of the generated workflow according to the first embodiment. As shown in FIG. 7, the underlined part is included in the reason. When the doctor touches the underlined part with the fingertip or move the cursor over the underlined part, the infarction region that is the reason will be highlighted in the image at the bottom of the screen.

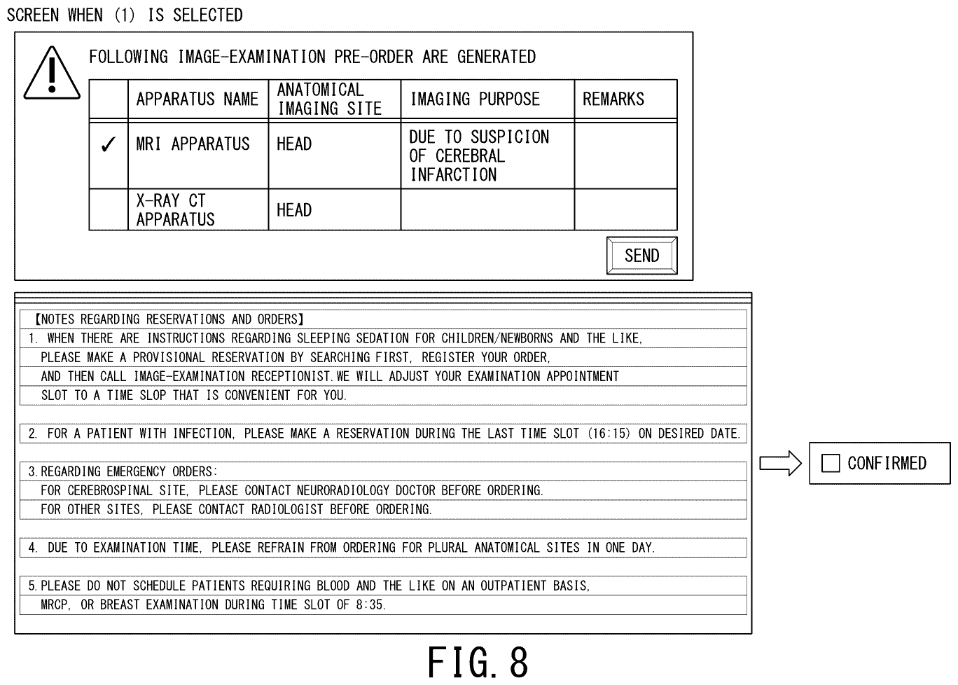

FIG. 8 illustrates a pre-order generation screen according to the first embodiment. FIG. 8 particularly shows one case of the pre-order generation screen when the option (1) of the workflows in FIG. 6 is selected. As shown in FIG. 8, for each image examination, a checkbox, an apparatus name, an anatomical imaging site, an imaging purpose, and remarks are displayed.

A check mark is entered in the checkbox in response to the doctor's selection of the apparatus name. The apparatus name is a name of the apparatus to be used for the image examination, and is set to be the MRI apparatus or the X-ray CT apparatus, for example. The anatomical imaging site is a part of the object to be imaged, and is set to be, for example, the head, the chest, or the abdomen. The imaging purpose is the purpose and reason for imaging the target part by using the apparatus, and is set as “because there is a suspicion of cerebral infarction”, for example. As to the column of the remarks, for example, notes on the object are set.

Furthermore, notes regarding reservations and orders are displayed at the bottom of the screen. The notes may include, for example, “Has the consent form for the contrast medium injection been obtained?”. A checkbox of “confirmed” is displayed on the right side of the notes. The send button is not activated unless the doctor enters a check mark in this checkbox. When the doctor touches the send button, the pre-order is issued from the medical information processing apparatus 2 to the hospital information system 3.

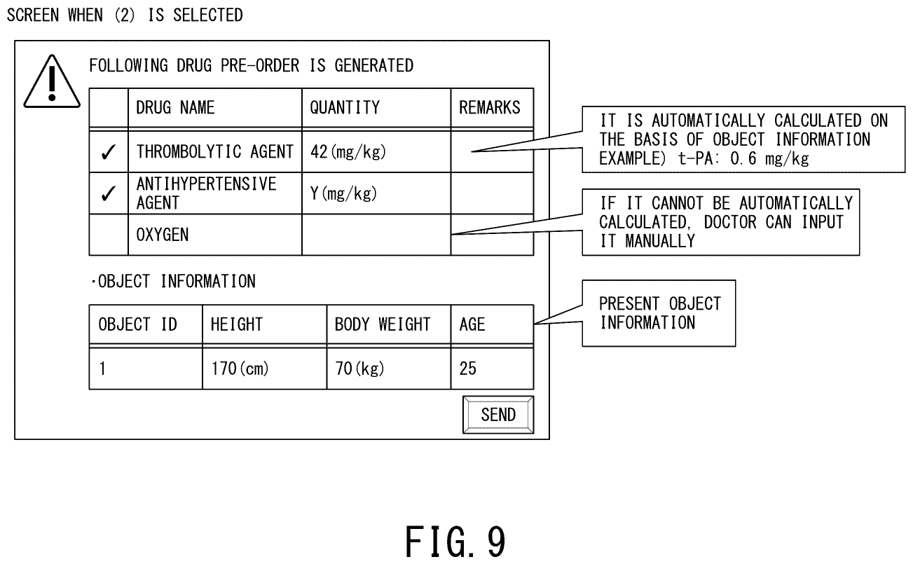

FIG. 9 illustrates another pre-order generation screen according to the first embodiment. FIG. 9 particularly illustrates one case of the pre-order generation screen when the option (2) of the workflows in FIG. 6 is selected. As shown in FIG. 9, a checkbox, a drug name, quantity, and remarks are displayed for each drug. A check mark is entered in the checkbox in accordance with the doctor's selection of the drug name. The drug name is the name of the drug administered to the object, such as a thrombolytic agent, an antihypertensive agent, and oxygen. The quantity is set to the quantity of the drug. As to the column of remarks, for example, notes on the object are set. Further, the object ID, height, weight, and age are set as the object information, for example.

For example, the quantity of the thrombolytic agent is automatically calculated on the basis of the object information. The quantity of oxygen is left blank, meaning it cannot be automatically calculated and can be entered manually by the doctor. The doctor can determine the appropriate quantity of the selected drug by referring to the object information. When the doctor enters a check mark in the checkbox of the required drug and clicks the send button, the pre-order is issued.

According to the first embodiment, the time and effort required for the doctors to generate an order can be reduced. Specifically, a subsequent imaging order or treatment order based on the attribute information of the object and the analysis information of the object can be proposed at the same time as presenting the finding image. The doctor can issue the order by approving the pre-order generated by the medical information processing apparatus 2 with one click on the mobile terminal 6 or the HIS terminal 7. Hence, the order can be executed quickly, and thus, the object can be examined and treated more quickly.

The mobile terminal 6 may transmit the formal order to the hospital information system 3. The medical information processing apparatus 2 may also transmit the formal order to the hospital information system 3.

### Second Embodiment

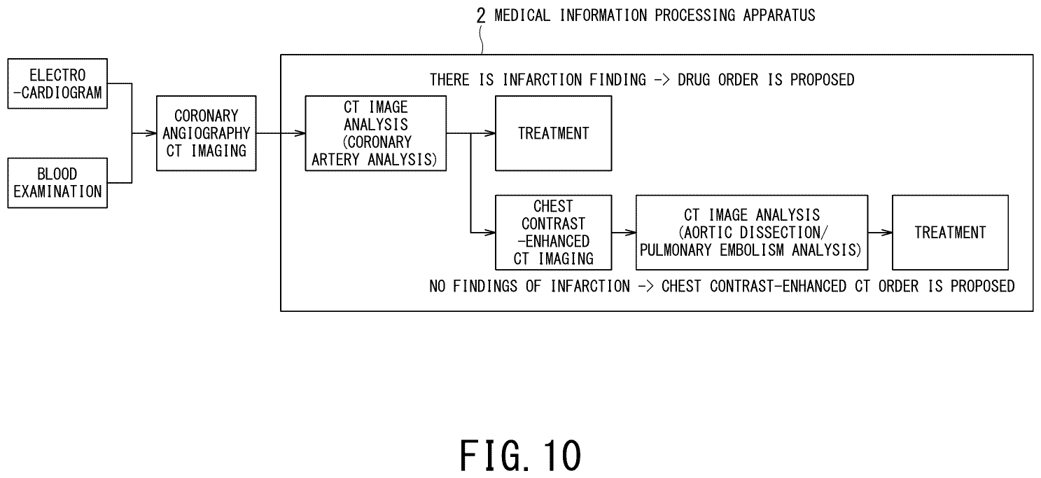

FIG. 10 illustrates a proposed workflow regarding an object with chest pain according to the second embodiment. In FIG. 10, it is assumed that the doctor initially suspects myocardial infarction of the object with chest pain on the basis of the electrocardiogram and the blood examination result of this object. In response to the imaging order from the doctor, the X-ray CT apparatus 41 performs coronary angiography CT imaging and transmits the CT images of this object to the medical information processing apparatus 2.

The medical information processing apparatus 2 receives the CT images of the object from the X-ray CT apparatus 41, and performs analysis of the CT images, especially the coronary artery analysis. If there is any finding of infarction in the coronary artery of the object based on a result of the coronary artery analysis, the medical information processing apparatus 2 proposes a drug order as the treatment order. If there is no finding of infarction in the coronary artery of the object based on a result of the coronary artery analysis, aortic dissection or pulmonary embolism is suspected, and thus, the medical information processing apparatus 2 proposes a chest contrast-enhanced CT imaging order as the imaging order. Further, the medical information processing apparatus 2 may propose a CT image analysis order as an analysis order and propose a treatment order.

According to at least one embodiment described above, the order can be quickly executed and the patient can be treated quickly.

While certain embodiments have been described, these embodiments have been presented by way of example only, and are not intended to limit the scope of the invention. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. A medical information processing apparatus comprising first processing circuitry configured to:
acquire attribute information indicating an attribute of an object and analysis information indicating an analysis result of a medical image of the object;
generate a pre-order including at least one of an imaging order and a treatment order based on the attribute information and the analysis information; and
cause a display to display the pre-order.

2. The medical information processing apparatus according to claim 1, wherein the first processing circuitry is configured to:
further generate rationale information indicating a grounds for generating the pre-order based on the attribute information and the analysis information; and
cause the display to display the rationale information together with the pre-order.

3. The medical information processing apparatus according to claim 1, wherein the first processing circuitry is configured to:
identify at least one candidate disease of the object based on the attribute information and the analysis information;
acquire history data of the imaging order or the treatment order, the imaging order being an instruction to generate a medical image necessary for diagnosing the candidate disease, the treatment order being an instruction to perform treatment of the candidate disease; and
generate the pre-order based on the history data.

4. The medical information processing apparatus according to claim 1, wherein the first processing circuitry is configured to:
generate a plurality of pre-orders; and
narrow down the pre-orders to be displayed on the display depending on number of times that each pre-order has been executed.

5. The medical information processing apparatus according to claim 1, wherein the analysis information is an analysis result obtained by analyzing the medical image by an image analysis application or a report made by an image interpretation doctor from the analysis result.

6. The medical information processing apparatus according to claim 1, wherein the first processing circuitry is configured to generate a plurality of pre-orders in cooperation with second processing circuitry of a hospital information management system, the second processing circuitry being configured to:
transmit an analysis order to the medical information processing apparatus, the analysis order being an instruction to analyze the medical image of the object and output the analysis result;
acquire medical history information indicating a medical history of the object;
acquire the pre-orders;
narrow down the pre-orders based on the medical history information to cause a display to display a narrowed-down pre-order;
receive approval of the narrowed-down pre-order by user's operation; and
issue at least one of the imaging order and the treatment order based on the pre-order of which the approval has been received.

7. A medical information processing system comprising a medical information processing apparatus and a hospital information management system,
wherein the medical information processing apparatus comprises first processing circuitry configured to:
output an analysis result of a medical image of an object by analyzing the medical image of the object in accordance with an analysis order received from the hospital information management system;
acquire attribute information indicating an attribute of the object and analysis information indicating the analysis result of the object;
generate a plurality of pre-orders including at least one of an imaging order and a treatment order based on the attribute information and the analysis information,
wherein the hospital information management system comprises second processing circuitry configured to:
transmit the analysis order to the medical information processing apparatus, the analysis order being an instruction to analyze the medical image of the object and output the analysis result;
acquire medical history information indicating a medical history of the object;
acquire the pre-orders;
narrow down the pre-orders based on the medical history information to cause a display to display a narrowed-down pre-order;
receive approval of the narrowed-down pre-order by user's operation; and
issue at least one of the imaging order and the treatment order based on the narrowed-down pre-order of which the approval has been received.

8. A medical information processing method to be executed by a medical information processing apparatus, the medical information processing method comprising steps of:
acquiring attribute information indicating an attribute of an object and analysis information indicating an analysis result of a medical image of the object;
generating a pre-order including at least one of an imaging order and a treatment order based on the attribute information and the analysis information; and
causing a display to display the pre-order.
